SEQUENCE LISTING (2 sequences)

SEQ ID NO: 1 - DNA, 2247 bp, Clostridium botulinum
  source 1..2247: mol_type = genomic DNA
atgacaaacctgaaaccctatataatatacgactggaaagaaaccatcctgaagaatagtaaggataactacagcattaacgagtcgattcccaagatattctctaaaaaaatctgcggaggacgtttttttaacagtaccttgagcggtaattggaaatcctggacgttgacagatgaaggtgaggggccacaccccgtcctgaaatgtaccatcgacaacgggtacttggaaatttatagcaacacttcgtcggagaagcactccctgaaagacatcgagatcaaagtgtgcatgtcgattaagccaaattcggacggcacccacagtttgtgcaaaaactctttttacataaagaccaatagtttaaagttgtctgaggatcgcttgattttatcccattgtttagataaattgattctggcgtggtttaaagacaaccacaaatacattgaattgttcataaaccgctcccgtatacaaacaagagtcgaaggagacctgagcttgttaggatgggacatagagtcttctgtctcttacaagactatgaacgagttcattaaaaaggacaatttatatgaaaagaagttccaccaatacatggaggtccggcggaatgaatacaccatcgatggtgagtttgggccctggcaaatgacgactggagccgacggtcagaacatccgtttcctttgtccgatcaaaagcgcaacgtataaaatcaatgatgatgtatatatcgcaaaacccgataactttataattatacaagtcgatttaaaatacttcgattccaagaccacaataatagacccatcaggtttaaataatgggcagcaattcaacctgaaagtgaagaccgatagtactgatgaaatcaatgctgtaatattggtaggttctagaatcactgatgtaaatgaagatctttatcccggagatgatgtctcattagagatagtcttcaagacctggtttaatgccaatatccagaaattcacccaaatattttcatacatccttctgaatgagaccagtaagattcccgaatatcaatggctgaaaccaacccagatctcgtacggttccgcttcagtaacgatgccagatccgagcaatcctaataaggagctgagcaaccttgacgcgtcgacgttcgcggctatggcaatggtggaaaatcacaaaaatgatcgcccgaaccatgccgtggataatcgtttcttggagttgtcgaaaacgcctgctgcgttcgctatctcaatgcccgaatttttgaaacatttcttggttacaggattgcaagccatgcagatcgacaacctggatgcgtttgaagtttcttccgagaacttagttataacgaacaaaaagaagatcaatttcgggaaaatacaggatcagaatagacaggtggatgccttgatcgagcctaataatttcaagcttgcgatacaaaataaccaggtagttgtcgaaattgttgacgcaacctggcaacaagtagttggggtcacaggtcacttcggctatagacaagcctacaatttaattctgaaaaatgaaaataatgtatacaaaccgatgctggaagagtcaggtgacgtaactataagctatatggtgaccgaggaggcatggaagacaactcaggacgctataataagtgcgaccgtagggttagttgtcgggaccattatcggaacggcattcagtaaacttagtgacaaactttataagtttctgaagagtaagttcatagtcaaaaacaaaaaggctagcctgaaaatatctggaaaggacataaatgaagtaatagaaatgtctgatatcagcaagccgcaactgctgtcgattaaaaaagctaatgctaagatttccaccgaagaagtagggctgatctctcagaatggatccacgagccttgaaaatcttgcaatattcaaaaataaaccccgtccgattggagagcgtgtgcaaattttagggctgaaattagtttcaggattgattacaacctttggctggagtataggcttcgtgttgcccgatatattgaaggacgtgatcaatgctaatataaacaacaactttgaagttttgccgggtatccaacaattcactcaacagtgtatcgggtcaattcagtggcctgataatagtgaactgaaaatcgattttgccaagttacagggcgtatatttgctggggggaaatctggtgaagatccccgagagcaactaa

SEQ ID NO: 2 - protein, 2244 aa, Clostridium botulinum
METTHRASNLEULYSPROTYRILEILETYRASPTRPLYSGLUTHRILELEULYSASNSERLYSASPASNTYRSERILEASNGLUSERILEPROLYSILEPHESERLYSLYSILECYSGLYGLYARGPHEPHEASNSERTHRLEUSERGLYASNTRPLYSSERTRPTHRLEUTHRASPGLUGLYGLUGLYPROHISPROVALLEULYSCYSTHRILEASPASNGLYTYRLEUGLUILETYRSERASNTHRSERSERGLULYSHISSERLEULYSASPILEGLUILELYSVALCYSMETSERILELYSPROASNSERASPGLYTHRHISSERLEUCYSLYSASNSERPHETYRILELYSTHRASNSERLEULYSLEUSERGLUASPARGLEUILELEUSERHISCYSLEUASPLYSLEUILELEUALATRPPHELYSASPASNHISLYSTYRILEGLULEUPHEILEASNARGSERARGILEGLNTHRARGVALGLUGLYASPLEUSERLEULEUGLYTRPASPILEGLUSERSERVALSERTYRLYSTHRMETASNGLUPHEILELYSLYSASPASNLEUTYRGLULYSLYSPHEHISGLNTYRMETGLUVALARGARGASNGLUTYRTHRILEASPGLYGLUPHEGLYPROTRPGLNMETTHRTHRGLYALAASPGLYGLNASNILEARGPHELEUCYSPROILELYSSERALATHRTYRLYSILEASNASPASPVALTYRILEALALYSPROASPASNPHEILEILEILEGLNVALASPLEULYSTYRPHEASPSERLYSTHRTHRILEILEASPPROSERGLYLEUASNASNGLYGLNGLNPHEASNLEULYSVALLYSTHRASPSERTHRASPGLUILEASNALAVALILELEUVALGLYSERARGILETHRASPVALASNGLUASPLEUTYRPROGLYASPASPVALSERLEUGLUILEVALPHELYSTHRTRPPHEASNALAASNILEGLNLYSPHETHRGLNILEPHESERTYRILELEULEUASNGLUTHRSERLYSILEPROGLUTYRGLNTRPLEULYSPROTHRGLNILESERTYRGLYSERALASERVALTHRMETPROASPPROSERASNPROASNLYSGLULEUSERASNLEUASPALASERTHRPHEALAALAMETALAMETVALGLUASNHISLYSASNASPARGPROASNHISALAVALASPASNARGPHELEUGLULEUSERLYSTHRPROALAALAPHEALAILESERMETPROGLUPHELEULYSHISPHELEUVALTHRGLYLEUGLNALAMETGLNILEASPASNLEUASPALAPHEGLUVALSERSERGLUASNLEUVALILETHRASNLYSLYSLYSILEASNPHEGLYLYSILEGLNASPGLNASNARGGLNVALASPALALEUILEGLUPROASNASNPHELYSLEUALAILEGLNASNASNGLNVALVALVALGLUILEVALASPALATHRTRPGLNGLNVALVALGLYVALTHRGLYHISPHEGLYTYRARGGLNALATYRASNLEUILELEULYSASNGLUASNASNVALTYRLYSPROMETLEUGLUGLUSERGLYASPVALTHRILESERTYRMETVALTHRGLUGLUALATRPLYSTHRTHRGLNASPALAILEILESERALATHRVALGLYLEUVALVALGLYTHRILEILEGLYTHRALAPHESERLYSLEUSERASPLYSLEUTYRLYSPHELEULYSSERLYSPHEILEVALLYSASNLYSLYSALASERLEULYSILESERGLYLYSASPILEASNGLUVALILEGLUMETSERASPILESERLYSPROGLNLEULEUSERILELYSLYSALAASNALALYSILESERTHRGLUGLUVALGLYLEUILESERGLNASNGLYSERTHRSERLEUGLUASNLEUALAILEPHELYSASNLYSPROARGPROILEGLYGLUARGVALGLNILELEUGLYLEULYSLEUVALSERGLYLEUILETHRTHRPHEGLYTRPSERILEGLYPHEVALLEUPROASPILELEULYSASPVALILEASNALAASNILEASNASNASNPHEGLUVALLEUPROGLYILEGLNGLNPHETHRGLNGLNCYSILEGLYSERILEGLNTRPPROASPASNSERGLULEULYSILEASPPHEALALYSLEUGLNGLYVALTYRLEULEUGLYGLYASNLEUVALLYSILEPROGLUSERASN

(19) United States
(12) Patent Application Publication
(10) Pub. No.: US 2026/0092087 A1
(43) Pub. Date: Apr. 2, 2026

(54) Method of Making a Pharmaceutical Composition Comprising a p80 Protein

(71) Applicant: Prime Bio, Inc., North Dartmouth, MA (US)

(72) Inventors: Bal Ram Singh, Rehoboth, MA (US); Raj Kumar, Dartmouth, MA (US)

(73) Assignee: Prime Bio, Inc., North Dartmouth, MA (US)

(21) Appl. No.: 19/352,451

(22) Filed: Oct. 7, 2025

Related U.S. Application Data
(62) Division of application No. 17/024,302, filed on Sep. 17, 2020, now Pat. No. 12,528,843.

(51) Int. Cl. C07K 14/33; A61K 38/00; A61K 45/06; C07K 1/18

(52) U.S. Cl. CPC C07K 14/33; A61K 45/06; C07K 1/18; A61K 38/00

(57) ABSTRACT

Present invention relates to a method of making a recombinant p80 polypeptide using an E. coli culture comprising a plurality of E. coli host cells each transformed or transfected with an expression vector containing a DNA fragment encoding a recombinant p80 polypeptide; and inducing the E. coli culture by adding a 0.2% L-Arabinose. The isolated, recombinant p80 is added to a pharmaceutical composition, wherein it binds to a therapeutic agent comprising one or more of: drugs, dyes, small molecules, biomolecules, proteins or a combination thereof. The p80 polypeptide enhances the transportation of the therapeutic agent into the bloodstream by the p80 functioning as a tight junction modulator to enhance the permeability of the intestinal epithelium to the therapeutic agent.

### CROSS REFERENCE TO RELATED APPLICATIONS

The present invention claims priority as a divisional to U.S. patent application Ser. No. 17/024,302 filed on Sep. 17, 2020, entitled “Pharmaceutical Composition Comprising P80 Protein”, which is hereby incorporated in its entirety.

### REFERENCE TO SEQUENCE LISTING SUBMITTED ELECTRONICALLY

This instant application contains a Sequence Listing, which has been submitted electronically in XML file format and is hereby incorporated by reference in its entirety. The XML sequence listing was created on Oct. 15, 2025, is named “P80 Sequence listing 07062025_Oct15.2025.xml” and is 5,710 bytes in size.

### FIELD OF THE INVENTION

The present invention relates to methods of isolating the p80 neurotoxin associated protein (NAP) from the Type E Clostridium botulinum neurotoxin (BoNT/E) complex. The p80 NAP is subsequently used in a pharmaceutical composition as a bioenhancer to increase the bioavailability of a drug it is bound to, via increasing absorption of the drug across the intestinal epithelium.

### BACKGROUND OF THE INVENTION

Several strains of an anaerobic bacteria belonging to the genus Clostridium, such as C. botulinum and C. baratii, have been widely studied for their ability to produce a neurotoxin known as the Botulinum Neurotoxin (BoNT). When humans or other animals come in contact with these neurotoxins, it is known to result in muscle paralysis by blocking the release of acetylcholine from cholinergic nerve endings (DasGupta et al. 1977). BoNTs have thus been classified by the Centers for Disease Control and Prevention (CDC) as one of the six highest-risk threat agents for bioterrorism (the “category A agents”) as a result of their extreme potency and lethality, the ease of production and transport, and the need for prolonged hospital intensive care upon exposure (Arnon et al. 2001). Eight antigenically different BONT serotypes, produced by C. botulinum, have been identified and designated as types A-H. The highly homologous primary structure of these serotypes forms the basis for the three functional domains of BoNT (Strotmeir et al. 2011, Montal 2010).

Seven of the serotypes (A-G) of botulinum neurotoxin (BoNT) are produced in the form of a complex with a group of neurotoxin associated proteins (NAPs) (Fujji 1993, Prabakaran et al. 2001) BONT is produced in three different progenitor toxin or complex types: M, L, and LL forms. There are variations in sizes and composition of complexes of different BONT serotypes (A through G). BoNT/A complex can exist in three forms: M, L or LL; BoNT/B, /C and /D complexes in two forms: Land M; BoNT/E and/F complexes are known to exist only in M form. The M form consists of neurotoxin (150 kDa) and a nontoxic protein component (120 kDa), which is called neurotoxin binding protein (NBP) (Singh et al. 1995a) or nontoxic, non-hemagglutinin component (NTNH) (East and Collins 1994) with 12S molecular size (the molecular size of complex forms is expressed as sedimentation equilibrium values). The L form has a molecular weight of about 500 kDa and a molecular size of 16S. The LL form is about 900 kDa and 19S. The Land LL complexes consist of several hemagglutinin components besides the BoNT and NBP, and exhibit hemagglutinin activity (Somers and DasGupta 1991; Fujii 1995).

In 1998, the Singh research group named the proteins other than the neurotoxin present in botulinum neurotoxin complex as neurotoxin associated proteins or NAPs (Fu et al. 1998). NAPs have at times been referred to as ANTPs (Associated Nontoxic Proteins) to emphasize their non-toxic property (Poulain et al. 2008). One type of NAP termed neurotoxin binding protein (NBP) is different from the other NAPs in that it clearly interacts with the neurotoxin portion of BoNT. For example, in the M form of BoNT, the 150 kDa BoNT (100 kDa heavy chain or HC and 50 kDa light chain or LC linked through a disulfide bond) is found in association with a ˜120 kDa NBP. NAPs present in BoNT/A comprise two main groups of proteins (Inoue et al., 1996 and Sharma etal., 2003): (i) Hemagglutinins (HA) of 17, 23, 33, 48 kDa; and (ii) Non-Toxin Non-Hemagglutinin of 138 kDa. While it is known that NAPs do not play a role in the toxin-induced blockade of cholinergic neurotransmission, they do however, play an importantrole in the protection of BoNTs against the proteases of the Gastrointestinal (GI) tract during oral poisoning. These proteins thus have the ability to enhance the oral toxicity of the neurotoxin significantly (Sakaguchi, 1982). Three of the HA NAPs are known to interact with intestinal epithelial cells and play an active role in BONT transport (Fujinaga et al. 1997; Fujinaga et al. 2004; Niwa et al. 2007). Strong support for the functional role of HAs in this regard is provided by the finding that HAs directly bind to E-cadherin and disrupt the intercellular epithelial barrier (Ito et al. 2011; Matsumura etal. 2008; Sugawara et al. 2010).

Of all the NAPs, the 33 kDa hemagglutinin (Hn33 or HA33) is the most abundant. Hn33 is a protease resistant and highly immunogenic type of NAPs that appears to play an important role in the translocation of the neurotoxin across the gut wall enhancing the endopeptidase activity of BoNT and protection of BoNT against proteases. The sequence homology among respective BoNTs and their NAPs range from 55.3% to 98.5%. Although it is believed that the proteins associated with other serotypes have protective effects (similar to that of type A) on the neurotoxin, not much is known about the structure-function of their NAPs.

According to Singh and Zhang (2009), type E Clostridium botulinum produces a complex similar to type A complex. Although it has been stated that BoNT/E neurotoxin was associated with one other protein (Sugii et al. 1983; Schantz et al. 1992; and Singh et al. 1995), a study by Singh & Zhang 2009, showed that the type E botulinum toxin exists in a complex that comprises the toxin and neurotoxin associated proteins. The five neurotoxin associated polypeptides were seen to have molecular weights of about 118, 80, 65, 40, and 18 kDa, respectively. The 118 kDa polypeptide is a well-known neurotoxin binding protein that was sequenced previously. The 80, 65, 40, and 18 kDa polypeptides, on the other hand are novel and have been partially sequenced by Singh & Zhang 2009. The present invention provides the structure and possible biological benefits of the 80 kDa BoNT Type E NAPs. This protein was selected as it was found to specifically bind to the Type E neurotoxin (Singh and Yang 2009). The study by Singh and Yang (2009) also revealed that the type E neurotoxin could bind directly to the 80 kDa type E neurotoxin associated protein, rather than associate indirectly with the neurotoxin via polypeptides in the complex. The potential of this polypeptide in disrupting the intercellular epithelial barrier is evidenced by using fluorescence microscopy and atomic force microscopy.

Conformational features of the 80 kDa NAPs were examined using circular dichroism and FTIR spectroscopy. These observations are helpful for explaining the various mechanistic processes of this molecule that enable the tight junction disruption that was observed. The secondary structure and tertiary structure were studied using circular dichroism (CD). The physiological activity of the protein was studied using intestinal epithelial cells (CaCo-2).

### SUMMARY OF THE INVENTION

The main aspect of the present invention is to provide a pharmaceutical composition comprising the p80 protein with one or more therapeutic agents, comprising: drugs, dyes, small molecules, biomolecules, proteins or a combination thereof.

In another aspect of the present invention, the p80 is obtained from BONT.

In another aspect of the present invention, the p80 is recombinant p80.

In another aspect, the nucleotide sequence of p80 is SEQ ID NO: 1.

In another aspect, the amino acid sequence of p80 is SEQ ID NO: 2. In another aspect of the present invention the BoNT is selected from type A-G.

In another aspect of the present invention, BoNT is type E (BoNT/E).

In another aspect, the p80 does not have hemagglutinin activity.

In another aspect of present invention, the p80 secondary structure is primarily a random coiled alpha (α) helix.

In another aspect of present invention, the pharmaceutical composition further comprises a microsphere encapsulating protein.

In another aspect of present invention, the pharmaceutical composition further comprises surface active agents, chelating agents, salicylates, anti-inflammatory agents, or phenothiazine. In another aspect of the invention, the pharmaceutical composition further comprises other associated proteins of the botulinum toxin complex.

In another aspect of the invention, the pharmaceutical composition further comprises other proteins of Clostridium Botulinum.

In another aspect of the invention is the enhanced bioavailability of the therapeutic agent, comprising drugs, dyes, small molecules, biomolecules, proteins or a mixture thereof, when administered with p80 or combinations thereof.

In another aspect of present invention, the pharmaceutical composition is in a lyophilized or gel form.

In another aspect, the pharmaceutical composition is stabilized at a pH in between 5.5 and 8.0.

Another aspect of present invention is to facilitate translocation of molecules associated with p80 or Hn across the intestinal epithelial layer.

In another aspect, the translocated molecule is a therapeutic agent comprising one or more of: drugs, dyes, small molecules, biomolecules, proteins or mixture thereof.

Another aspect of the present invention is providing a recombinant p80, an 80 kDa protein.

Another aspect of present invention is to provide a process for the preparation of recombinant p80, an 80 kDa protein.

Another aspect of present invention is a process to isolate and purify p80 from BONT.

Another aspect of the present invention is to provide a process for the preparation of recombinant p80.

In another aspect of the invention is that p80 protein does not show cytotoxicity up to a concentration of 300 UM when treated for 24 hours.

In another aspect a process for the preparation of recombinant p80, comprising the steps of
a. Induction of E. coli culture OD₆₀₀=0.6-0.8 by adding 0.2% L-Arabinose;
b. Growing the culture at 20° C. for 16-18 hrs;
c. Lysis of the cell paste;
d. Centrifuging the culture;
e. Supernatant is collected;
f. Supernatant is purified using Ni-NTA affinity column;
g. Combine the pool after elution using 250 mM imidazole,
h. Concentrate the pool by using Centriprep-30; and
i. Purifying using anion exchange column.

In another aspect of the process, the BoNT is BoNT/E.

According to another aspect, the present invention comprises a method of treatment by administrating an effective amount of a pharmaceutical composition of comprising p80 protein with drugs, dyes, small molecules, biomolecules, proteins or combinations thereof.

In another aspect of the present invention, the method of administration is an oral route, sublingual and buccal routes, rectal route, vaginal route, ocular route, optic route or nasal route.

### BRIEF DESCRIPTION OF THE DRAWINGS

FIG. 1: Elution profile of P80 from DEAE sephadex A 50 column. Second peak was p80. Molecular weight and purity were confirmed for the selected fractions by running 4-20% SDS-PAGE gel. Fraction numbers 50 to 66 were collected and pooled after checking the purity with SDS-PAGE.

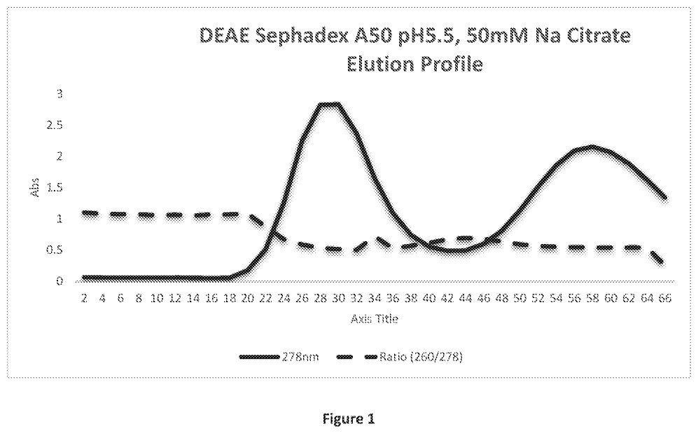

FIG. 2: Translocation of DrBoNT/a in the presence of p80 across the epithelial cell lines Caco 2. A constant amount of DrBoNT/A-488 (200 nM was mixed with serum free media containing 200 nM of P80 and added to the apical side of the transwell insert). After incubating at 37° C., 5% CO₂, for 2, 4, 6, 8, 24 h, aliquots of basal medium were removed, and fluorescence of the sample was measured using a plate reader. Measurement was measured in triplicate and fold change with respect to 0 hr was plotted against the treatment and time.

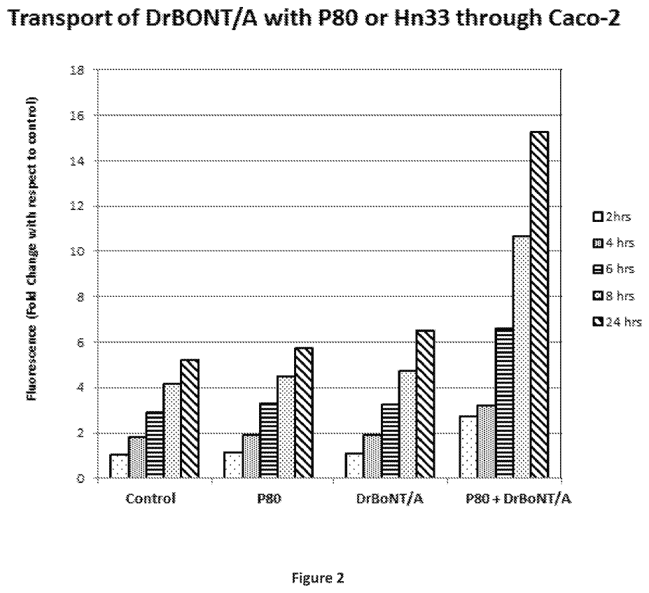

### DETAILED DESCRIPTION OF THE INVENTION

Type E Botulinum Neurotoxin (BoNT/E) is a large protein toxin (approximately 150 kDa) that is able to pass through the epithelial barrier in the digestive tract. This toxin is produced by Clostridium botulinum along with five neurotoxin associated polypeptides (NAPs). Some of these proteins play an important role in the mechanism by which this large protein toxin crosses the intestinal epithelial barrier. The NAP found to specifically bind to the Type E neurotoxin has been purified as an 80 kDa protein (p80). The present invention provides the structure of the p80 protein as well as its interaction with the intestinal epithelial barrier using a cell-based model. These in vitro experiments have proved the potential of the p80 to facilitate an increased absorption of BoNT through the paracellular route of the intestinal epithelium. Therefore, p80 could be used as a bioenhancer to improve the a drug's bioavailability, i.e. through the supplementation of the main therapeutic agent with a secondary agent (e.g. p80) to improve the rate of the drug going across intestinal epithelial barrier. The present invention provides a composition with p80, which is used as the bioenhancer for the therapeutic agent.

In the present application, the protein p80 was purified from the BoNT Type E complex in two steps, using DEAE sephandex A-50 anion exchange column followed by CM Sepharose CL-6B cation exchange column to obtain pure P80 protein. Secondary structure estimates showed p80 to be predominantly an alpha (α)-helical protein mixed with a random coil. This was consistently observed not only from thespectral regions of FT-IR, but also from an additional independenttechnique (far-UV CD). The protein did not show any cytotoxicity even at high concentrations of 300 nM, when treated for 24 hours. Unlike other NAPs, Hn-33 from type A BoNT, p80 did not show any hemagglutinin activity. This indicates an inability of p80 to bind to specific sugars, a biochemical process involved in cell recognition. In vitro analysis of intestinal epithelial cells was carried out using various microscopic techniques in order to study their potential as a bioenhancer. The p80 and Hn33 treated cells increased localization of the marker molecule along the tight junctions of cells, although to different extents, indicating possible increase of paracellular transportation. The experiments carried out to measure the rate of translocation helped quantitatethe rate of translocation of the co-incubated therapeutic molecule (DrBoNT) and indicated a greater rate of translocation in the p80 treated cells when compared to the untreated of Hn33 cells. This evidenced that treated cells alter the extent of trafficking of marker molecules that are co-incubated with the p80. The topographical images obtained through the AFM analysis allowed us to see an increased cell-cell separation as a result of Hn33/p80 treatment on a cell monolayer. The data further supports the paracellular transport in p80 when compared to the Hn33 treatment and even provides a better visual of the changes that the cell might have undergone along its tight junction with time. These images clearly showed separation of cells along the tight junction. On the other hand, the immunofluorescence assays indicated an increased rearrangement of actin cytoskeletal elements in Hn33 treated cells when compared to p80, indicating an alternative mechanism of action of these proteins.

What is claimed is:

1. A process for the preparation of a recombinant p80 polypeptide, comprising at least the steps of:
a) providing an E. coli culture comprising a plurality of E. coli host cells each transformed with an expression vector containing a DNA fragment encoding a recombinant p80 polypeptide;
b) inducing the E. coli culture by adding a 0.2% L-Arabinose;
c) growing the E. coli culture at 20° C. for 16-18 hr to produce a cell paste;
d) lysing the cell paste;
e) centrifuging the cell paste, and collecting a supernatant from the cell paste;
f) purifying the cell paste using a Ni-NTA affinity column to produce a pool;
g) combining the pool after elution using 250 mM imidazole, and concentrating the pool by using Centriprep-30; and
h) purifying a plurality of p80 polypeptides from the pool using an anion exchange column.

2. The process of claim 1, wherein the DNA fragment encoded in the E. coli expression vector comprises the p80 polynucleotide sequence of SEQ ID NO: 1.

3. The process of claim 1, wherein the step (h) each of the plurality of p80 polypeptides purified from the anion exchange column comprises the amino acid sequence of SEQ ID NO: 2.

4. The process of claim 1, wherein inducing of the E. coli culture occurs when the E. coli culture obtains at an optical density of 600 nm (OD600), and a reading between 0.6 and 0.8.

5. The process of claim 1, wherein each of the recombinant p80 polypeptides are added to a pharmaceutical composition comprising a therapeutic agent.

6. The process of claim 5, wherein the therapeutic agent comprises one or more of: drugs, dyes, small molecules, biomolecules, proteins or a combination thereof.

7. The process of claim 5, wherein each of the plurality of recombinant p80 polypeptides binds with the therapeutic agent to increase the bioavailability of the therapeutic agent in the bloodstream.

8. The process of claim 7, wherein increased bioavailability of the therapeutic agents occurs after the plurality of recombinant p80 polypeptides bind to and expand the opening of a plurality of tight junctions on the surface of a user's intestinal epithelium.

9. The process of claim 8, wherein the therapeutic agent passes from the intestinal epithelium and into the bloodstream at a higher rate and concentration as compared to a pharmaceutical composition comprising the therapeutic agent and excluding the plurality of recombinant p80 polypeptides.
